(19) United States
(12) Patent Application Publication
(10) Pub. No.: US 2020/0069567 A1
(43) Pub. Date: Mar. 5, 2020

(54) Tattoo Ink Containing Cannabis or Hemp Derived Cannabinoids or Mixture of Both

(71) Applicant: Greg Rubin, Woodland Hills, CA (US)

(72) Inventor: Greg Rubin, Woodland Hills, CA (US)

(21) Appl. No.: 16/560,914

(22) Filed: Sep. 4, 2019

Related U.S. Application Data
(60) Provisional application No. 62/726,824, filed on Sep. 4, 2018.

(51) Int. Cl. A61K 8/9789; A61Q 1/02

(52) U.S. Cl. CPC A61K 8/9789; A61K 2800/43; A61Q 1/025

(57) ABSTRACT

A composition of matter having a tattoo ink, one or more tattoo pigments, and at least one cannabinoid extracted from a cannabis plant.

### CROSS-REFERENCE TO RELATED APPLICATIONS

This application claims the benefit of U.S. Provisional Application No. 62/726,824, filed Sep. 4, 2018, which is hereby incorporated by reference, to the extent that it is not conflicting with the present application.

### FIELD OF THE INVENTION

The present invention relates generally to liquid inks. More specifically, the present invention relates to tattoo inks that are mixed with tattoo pigments and at least one cannabinoid extracted from the cannabis plant.

### BACKGROUND OF THE INVENTION

Tattoos have been part of human culture for over five thousand years. A mummified body was discovered to be a hunter dated back to 5,300 years ago, and 61 tattoos were found on his body near the joints. Perhaps tattooing was used as an ancient treatment for arthritis. Placing a tattoo on a skin requires putting tattoo colorants on needles, and the needles puncture the skin to deliver the colorants to the bottom layer of the skin, known as the dermis. Skin puncture from needles creates pain, inflammation, and risk of infection.

Skin puncture causes local cells to release prostaglandins, chemicals responsible for inflammation and pain. Additionally, nociceptors nearby also release substance P, a neurotransmitter transmitting pain signal to the brain. When an area of the skin is punctured, foreign microbes can penetrate into the area, leading to a potential infection.

The present invention reduces the pain, inflammation, and risk of infection from tattooing. Specifically, the present invention is a tattoo ink that have antibiotic, anti-inflammatory, and pain-killing properties by combining a tattoo colorant with certain chemicals known to have one or more of the three properties. Scientific literature suggests that members of the cannabinoids, chemical compounds from the cannabis tattoo inks, wherein the tattoo inks can reduce the risk of infection and can alleviate the pain and inflammation during a tattooing.

Cannabis is a plant having over 113 identified cannabinoids. Members of the family of cannabinoids include tetrahydrocannabinol (THC). THC has been known to have anti-inflammatory properties. Another member of the cannabinoids is cannabidiol (CBD). CBD has been known to have pain-killing properties. Additionally, studies have found that TCH and CBD can have antibiotic properties. More studies are being conducted to find out additional beneficial properties of cannabinoids. Thus, combining one or more cannabinoids to a tattoo colorant can create a tattoo ink that minimizes pain, inflammation, and infection.

### SUMMARY OF THE INVENTION

In an embodiment, a composition of matter is provided, the composition comprising a tattoo ink, one or more tattoo pigments, and at least one cannabinoid extracted from a cannabis plant.

### DETAIL DESCRIPTIONS OF THE INVENTION

The present invention is a tattoo ink comprising a tattoo colorant, a carrier solution, and at least one cannabinoid. In an alternative embodiment, the present invention is a tattoo ink comprising a tattoo colorant, a carrier solution, at least one cannabinoid, and at least one emulsifying agent.

A tattoo colorant is a pigment suspended in a carrier solution. Tattoo pigments are metal salts, plastics, or vegetable dyes. A pigment provides a color to a tattoo. A tattoo colorant with a black color is from iron oxide (Fe3O4 or FeO), carbon, or logwood. A tattoo colorant with a brown color is from ochre, iron (ferric) oxides mixed with clay. To create a red color, a tattoo colorant is from cinnabar, cadmium red, iron oxide (Fe2O3), or napthol-AS pigment. For an orange color, a tattoo colorant is from disazodiarylide, disazopyrazolone, or cadmium selenosulfide. A yellow color means that a tattoo colorant is from cadmium yellow, ochres, curcuma yellow, chrome yellow, or disazodiarylide. A green color means that a tattoo colorant is from chromium oxide (Cr2O3), anadomis green, malachite, ferrocyanides, lead chromate, monoazo pigment, Cu/Al phthalocyanine, or Cu phthalocyanine. To have a blue color, a tattoo colorant is from azure blue, cobalt blue or Cu-phthalocyanine. A violet color means that atattoo colorant is from manganese ammonium pyrophosphate, various aluminum salts, quinacridone, dioxazine or carbazole. Finally, to make a white color, a tattoo colorant is from lead carbonate, titanium dioxide, barium sulfate, or zinc oxide.

A carrier is a fluid used to transport a tattoo colorant to the dermis of the skin. A carrier is a solvent used to dissolve a tattoo colorant to form a solution, wherein the solution carries the colorant from needles to the dermis. A carrier encapsulates, encircles, surrounds, links, or connects to a tattoo colorant. A typical carrier is ethyl alcohol, denatured alcohols, water, methanol, rubbing alcohol, propylene glycol, or glycerine.

A cannabinoid is a chemical compound derived from the cannabis plant. More than 100 cannabinoids have been identified from the cannabis plant. Preferred cannabinoids are tetrahydrocannabinol (THC), tetrahydrocannabinolic acid (THCA), cannabidiol (CBD), cannabidiolic acid (CBDA), cannabinol (CBN), cannabichromene (CBC), cannabigerol (CBG), cannabicyclol (CBL), cannabivarin (CBV), tetrahydrocannbivarin (THCV), cannabidivarin (CBDV), cannabichromevarin (CBCV), cannabigerovarin (CBGV), cannabigerol monomethyl ether (CBGM), cannabielsoin (CBE) or cannabicitran (CBT). In a first preferred embodiment, at least one cannabinoid dissolves into a solvent, wherein the cannabinoid joins, links, connects, encapsulates, surrounds, or encircles a tattoo colorant. In a second preferred embodiment, an emulsifying agent joins the cannabinoid to the solution that dissolves the colorant, wherein the mixed product is an emulsion.

The emulsifying agent is a chemical compound soluble in both fat and water, and the agents enable fat to be uniformly dispersed in water as an emulsion. The potential list of emulsifying agents includes, but not limited to, agar, albumin, alginates, caein, ceatyl alcohol, cholic acid, desoxycholic acid, diacetyl tartaric acid esters, egg yolk, glycerol, gums, Irish moss, lecithin, monoglycerides, diglycerides, monosodium phosphate, monostearate, ox bile extract, propylene glycol, soaps, and taurocholic acid.

The present invention has a plurality of ratios from the combination of a tattoo colorant, carrier, cannabinoids, and emulsifying agents. The present invention can be a form of a cream, an ointment, a paste, a lotion, or a gel.

Thus, the present invention is a tattoo ink that has a mixture of a tattoo colorant and one or more cannabinoids.

Although the invention has been explained in relation to its preferred embodiment, it is to be understood that many other possible modifications and variations can be made without departing from the spirit and scope of the invention.

What is claimed is:

1. A composition of matter comprising a tattoo ink, one or more tattoo pigments, and at least one cannabinoid extracted from a cannabis plant.
